(19) United States
(12) Patent Application Publication
(10) Pub. No.: US 2024/0091498 A1
(43) Pub. Date: Mar. 21, 2024

(54) THERAPEUTIC AGENT DELIVERY CATHETER SYSTEMS

(71) Applicant: THE REGENTS OF THE UNIVERSITY OF CALIFORNIA, Oakland, CA (US)

(72) Inventors: Tony L. Yaksh, San Diego, CA (US); Matthew Hunt, Oak Park, CA (US); Kelly Eddinger, La Jolla, CA (US)

(21) Appl. No.: 18/256,720

(22) PCT Filed: Nov. 30, 2021
(86) PCT No.: PCT/US2021/061101

§ 371 Date: Jun. 9, 2023

Related U.S. Application Data
(60) Provisional application No. 63/124,399, filed on Dec. 11, 2020.

(51) Int. Cl. A61M 25/00

(52) U.S. Cl. CPC A61M 25/007; A61M 2210/1089

(57) ABSTRACT

A therapeutic agent delivery catheter system includes an elongate tube defining a lumen therein formed from biocompatible material and sized to insert in a patient. The elongate tube has a length that extends to reach a treatment region of the patient. A pattern of slits forming microvalves penetrates an outer wall of the tube. The slits are small enough to remain closed to contain a solution of therapeutic agent, and are configured to open in response to a pressure pulse in the lumen and emit a microjet of therapeutic agent from each slit in the pattern of slits distributed along the rostro caudal extent of the implanted catheter.

### PRIORITY CLAIM AND REFERENCE TO RELATED APPLICATION

The application claims priority under 35 U.S.C. § 119 and all applicable statutes and treaties from prior U.S. provisional application Ser. No. 63/124,399 which was filed Dec. 11, 2020.

### FIELD

A field of the invention is catheters for the delivery of therapeutic agents. Catheter systems of the invention are applicable to delivery of therapeutic agents to biological spaces in which drug distribution in the local volume of tissue is restricted or there is little flow, such as the subcutaneous, intramuscular, epidural or intrathecal spaces.

### BACKGROUND

Spinal therapeutic agents may be administered chronically to modify pain, spasticity and neurodegenerative disorders. Therapeutic agents delivered into the intrathecal space must undergo distribution from the catheter to avoid pooling of injectate at the catheter tip, avoiding local toxicity secondary to the exposure of local tissues to high injectate concentrations and must be distributed rostrocaudally to the target sites of action, which in the average human readily spans a 45 cm rostral-caudal distance.

Achieving these goals is complicated by the need to use small intrathecal injectate volumes and the fact that there is little CSF flow. These factors can result in therapeutic agents accumulating immediately proximal to the catheter tip. Such accumulation results in the need to increase drug doses to amplify local mixing and rostrocaudal distribution. In addition, poor mixing enhances the risk of local spinal toxicity which is dependent for many therapeutics upon local injectate concentrations.

Conventional spinal (intrathecal) catheters for therapeutic delivery include a large bore catheter with several large exit ports near the tip. With these catheters, because of the small volume or the low rate of delivery, as when used with an implanted pump, the injectate typically exits at low velocities, often from the first port only. This low exit velocity results in the spinal tissue immediately adjacent to the first port to be exposed to high local injectate concentrations. In the case of opiates this leads to a well-defined toxicity (the meningeal granuloma), which can be reduced by lowering drug concentrations or increasing dilutions, a strategy limited by the restricted pump reservoir volume.

There are several commercial spinal catheters: the Medtronic Ascenda and the Braun Cellsite. Each of these includes relatively large open ports, which are distributed along the distal end of the catheter.

U.S. Pat. No. 7,232,425 discloses a catheter for drug delivery via diffusion and dispersion of medication along the tubular element's length near a distal discharge port through disparate perforations, which may be formed as striations of increasing length nearer the distal discharge port. The perforations are open and are arranged with different sized openings which are said to provide a uniform volumetric discharge of therapeutic fluids injected through the lumen and into a treatment zone.

The above catheter designs suffer from two major issues. i) the use of small open ports for delivery do not allow parsing the delivery of a small volume over a long length of catheter, as the pressure gradient produced will lead to movement of the majority of injectate though the proximal ports, no matter how small, with minimal pressure being available to drive fluid from the distal ports. ii) With conventional open port catheters, in the absence of therapeutic fluid flow, there cellular debris, fibroblasts or protein accumulation will occur within the open catheter lumen. This presents problems for chronically implanted systems that may be injected with therapeutic agent intermittently.

### SUMMARY OF THE INVENTION

A preferred embodiment provides a therapeutic agent delivery catheter system includes an elongate tube defining a lumen therein formed from biocompatible material and sized to insert in a patient. The elongate tube has a length that extends to reach a treatment region of the patient. A pattern of slits penetrates an outer wall of the tube. The slits are small enough to remain closed to contain a solution of therapeutic agent, and are configured to open in response to a pressure pulse in the lumen and emit a microjet of therapeutic agent from each slit in the pattern of slits.

### BRIEF DESCRIPTION OF THE DRAWINGS

FIGS. 1A-1C are respective side partial, cross-section and perspective particle views of a preferred therapeutic agent delivery catheter system with exemplary inner and outer diameters and exemplary valve position and spacing dimensions;

FIG. 2 is an image series over a 0.7 second showing operation of a single microvalve in an experimental therapeutic agent delivery catheter system opening in a shallow diffusion chamber in response to a pressure pulse in its lumen; and

FIGS. 3A-3D are data and images of an experiment demonstrated paired valve catheter distribution of blue dye from an experimental therapeutic agent delivery catheter system in the diffusion chamber; FIG. 3A analyzes 10 μL of blue dye injected at 1 ml per minute, with paired ports numbered 1-5 show dye exiting with high velocity in opposite directions; FIG. 3B is an analysis of images are subtracted from pre-injection conditions, inverted, of average dye intensity over time in the diffusion chamber; FIGS. 3C and 3D high magnification images respectively showing a closed and open microvalve.

### DETAILED DESCRIPTION OF THE PREFERRED EMBODIMENTS

A preferred embodiment therapeutic agent delivery catheter system includes an elongate tube defining a lumen therein formed from biocompatible material and sized to insert in a patient. A preferred embodiment therapeutic agent delivery catheter system includes an elongate tube defining a lumen therein formed from biocompatible material and sized to insert in a patient. The elongate tube has a length that extends to reach a treatment region of the patient. A pattern of slits penetrates an outer wall of the tube. The slits are small enough to remain closed to contain a solution of therapeutic agent, and are configured to open in response to a pressure pulse in the lumen and thereby emit a microjet of therapeutic agent from each slit in the pattern of slits.

Preferred embodiments described below focus on delivery of therapeutics into the intrathecal fluid space (cerebrospinal fluid). The invention is widely applicable, however, to provide a distributed delivery of small injectate volumes into many organ systems. For example, subcutaneous delivery of analgesics and anesthetics into a wound margin or adjacent to a nerve for anesthesia, into a tumor bed for anticancer therapy, into brain parenchyma to deliver therapeutics for treatment of a large volume of brain tissue as required in neurodegenerative disorders, into a vascular bed to provide broad exposure of a length of blood vessel/or tissue. In each example, the utilization of the catheter system requires a distributed delivery of small volume in large volumes of tissue and the need to have a chronic system that can be tolerated for extended periods without the risk accumulation of debris and cellular ingrowth when used only periodically. These issues are specifically addressed by this invention.

The slits are preferably evenly distributed along the catheter at a distance determined by the treatment region. The pattern preferably arranges slits at different radial angles around a circumference of the elongate tube. One or more X-ray markers can be included aid in positioning the pattern of slits A system of the invention connects to a pump and control system that is configured to maintain fluid pressure in the delivery of the pressure pulse. As MPVs cause a pressure pulse in the closed elongate tube to be evenly distributed along the rostrocaudal length of the fluid filled tube, when the pressure is sufficient to cause the MPV to open, it does so at concurrently at all MPVs along the rostrocaudal extent of the tube. Accordingly, the same volume of fluid exits as a high velocity microjet at all MPVs at the same time regardless of their distance for the proximal end of the catheter. This principle distinguishes the properties of the MPV catheter from a catheter with a series of open ports along its length

The length of the slit for any given catheter will determine the opening pressure and will be defined by the ability of the delivery systems to exceed the opening pressure. The pressure defining the opening of the MPV and thereby the formation of a microjet will vary by the physical characteristics of the catheter and the wall thickness. The optimal slit length for any catheter (varying in size, wall thickness and composition) can thus be readily ascertained in an experimental setting with a diffusion chamber by someone skilled in the art of assessing fluid movement in a shallow fluid filled diffusion chamber with a marker dye and video imaging.

In an example application, the elongate tube is sized for intrathecal treatment and the pattern locates and arranges slits along a rostro-caudal length. The elongate tube includes a pattern of slits that form micro-poly-valves (MPVs) distributed along a longitudinal length of the catheter. For example, the distribution of MPVs can correspond to the rostro-caudal length. The MPVs are sized to remain closed while providing an even delivery of small volumes of infusate over long lengths of the spinal cord (or another biological target/space) and enhanced bilateral distribution from the catheter. Preferred catheters can lower therapeutic dose requirements and enhance safety, avoiding local toxicity related to sustained concentrations proximal to the catheter tip of conventional catheters, which risk serious adverse effects.

The preferred catheters thus provides a closed system when there is no injection. This property precludes accumulation of foreign material in the catheter. This property is essential for the use of these catheters for long term placement, particularly when drugs are to be delivered intermittently.

Suitable catheter material include conventional biocompatible materials used in prior catheters. Example materials include those which have been approved as being biocompatible, including polyurethane, polyethylene or silicone, or other material of appropriate, flexibility implant compatibility and durability in single or multiple layers.

Preferred catheters of the invention can be paired with a subcutaneous port to allow percutaneous access to the catheter for bolus delivery or to a pump delivery system. For long term use, the pump system can be implanted in the body. The only stipulation as to the pump properties is that it is able to generate pressure sufficient to open the MPV valves when a therapeutic agent is to be delivered such as via a delivery pulse.

Preferred embodiments of the invention will now be discussed with respect to experiments and drawings. Broader aspects of the invention will be understood by artisans in view of the general knowledge in the art and the description of the experiments that follows.

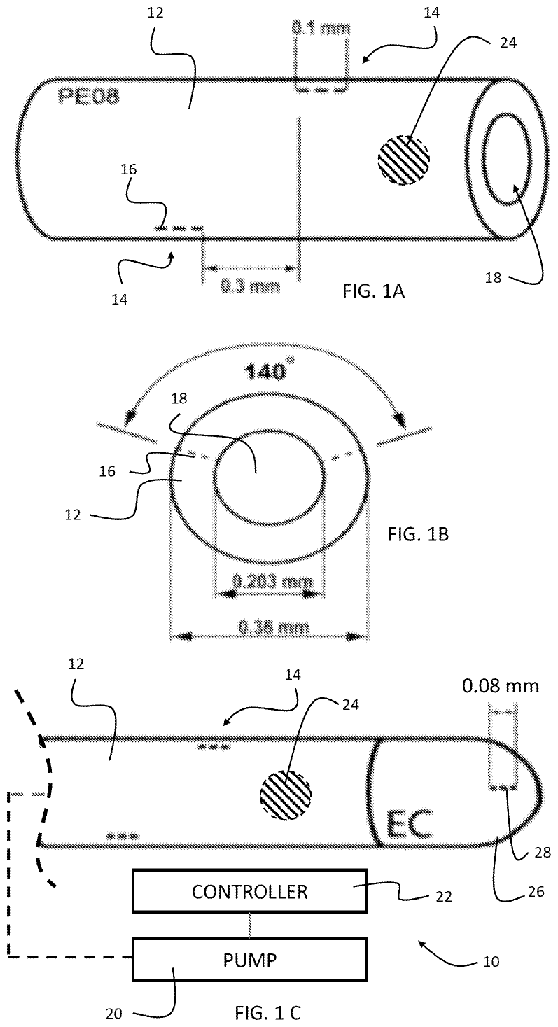

FIGS. 1A-1C show a preferred embodiment therapeutic agent delivery catheter system 10 with an elongate tube 12 defining a lumen 14. The tube 12 is formed from biocompatible material and sized to insert in a patient. The materials can be material conventionally used in medical catheters approved for human use, e.g., thermoplastic elastomers (TPE) such as polyether block amide (PEBA) olefin-based TPE, and poly(methylvinylidene cyanide)(PMVC) or medical grade polymers available from various manufacturers. Many such materials can be engineered to different flexibility and strengths to traverse a body lumen and reach a treatment region as is known in the art.

The diameter of the catheter MPV employed will be optimally determined by the clinical purpose for which the distributed delivery of fluids is to be employed, as for example nominally ranging in size from urethral catheters (4-7 mm), cardiac catheters (1-5 mm), venous catheters (2-4 mm), subcutaneous catheters for delivery to wound margins or perineurally (1-2 mm); intracranial catheters for parenchymal infusion (0.5-1.0 mm), intrathecal catheters-large (1-1.5 mm) and intrathecal catheters-microbore (0.3-1.0 mm) Indicated sizes are nominal and given as being representative of the range of diameters commonly employed for the respective application.

In the present description we present, as a teaching example, preparation and properties of a MPV system for the most challenging application of the present approach using a catheter of small diameter (0.36 mm) referred to here as PE-08.

The elongated tube 12 has a length that extends to reach a treatment region of the patient from a conventional entry point for minimally invasive insertion in the patient. A pattern 14 of slits 16 penetrates an outer wall of the tube, preferably such that the slits 16 are wherein the slits are evenly distributed along a longitudinal axis of the catheter over a distance determined by a treatment region of the patient. The slits 16 being are small enough to remain reliably closed prior to fluid delivery, such as during insertion of the tube into the patient and when its lumen 18 is initially filled with a solution of therapeutic agent, and open in response to a pressure pulse in the lumen 18, causing the emission of a microjet of therapeutic agent solution from each slit 16 in the pattern 14 of slits 16. For a particular pattern of slits, the pressure threshold can be determined experimentally, and the pressure pulse to emit can be controlled via the rate of delivery of a manual bolus, or the rate of delivery by a pump (such as an external syringe pump or an implanted pumping device) 20 and a controller 22. Insertion of the elongated tube 12 can be via a conventional approach with a percutaneous needle, such as used in typical medical catheter delivery systems. One or more X-ray markers 24 can be included in or on the tube 12 to aid in placement of the tube 12 in a treatment region of a patient. A distal tip 26 of the elongate catheter tube 12 is rounded and includes an additional microvalve slit 28, which is preferably a slightly smaller slit than the other slits 16, e.g. the slits 16 can be 0.1 mm long and the slit 28 can be 0.08 mm long. The distant slit acts as a release valve for any gases trapped in the catheter.

A preferred therapeutic catheter includes a pattern of multiple paired-valves with each valve consisting of one slit (as indicated in FIGS. 1A and 1C). Microvalves oriented parallel to the catheter longitudinal axis are created in pairs, preferably at an angle of 120° to 180° and staggered at varying distances between them in the longitudinal axis, with longitudinally adjacent slits being radially offset from each other. Microvalves are made as slits without removing any material from the catheter and thus critically forming a fluid tight seal when closed. As an example, microvalve sizes and distribution, as described, are optimized for a PE-08 polyethylene catheter that has a delivery distribution of approximately 8-10 cm in length. In this expression of the devices, Each of the pair of microvalves is staggered at a longitudinal distance from each other of 0.3 mm

The valves are situated over longitudinal length corresponding to a treatment area, e.g. along the proximal-distal extent of the catheter. The distribution of these microvalves defines the distribution of drug delivery sites in the adjacent tissues. The longitudinal distance between valves is small, but closely proximate valves are preferably radially offset from each other, e.g. by ˜140° from one another, as shown in FIG. 1B. The proximal-distal length can be determined according to a patient to be treated, based upon measurements, and a catheter length and length of MPV distribution can be set according to patient anatomy and tissues to which distributed delivery is desired.

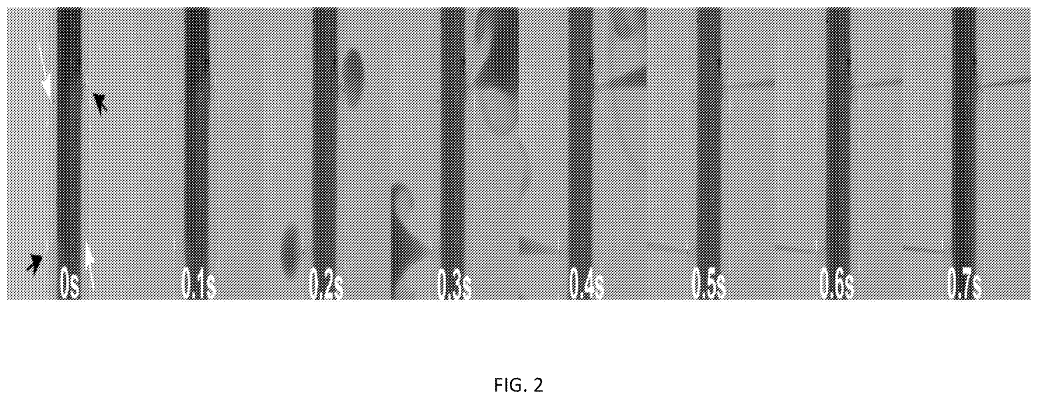

FIG. 1B exemplary dimensions were used in an experimental prototype that was tested, and images of the testing over a period of 0.7 seconds as a pressure pulse was applied are show in FIG. 2. The microvalves consist of small slits that were formed in an elongate catheter tube without removing any material, which allows the valves to self-seal but open under pressure. As shown in FIG. 2, a pressure pulse creates a significant high velocity solution jet that extends away from the wall of the catheter and serves to dilute the local infused volume of injectate in a larger local volume.

The small slits provide a high impedance exit, so the pressure gradient along the entire length of the catheter is the same. Hence the transmural gradient is the same at each valve. Thus, the injectate exiting each valve, whether proximal or distal to the drug delivery source (pump/syringe), will be the same. The total injectate is thus evenly distributed over microvalves located along the catheter length. Testing has demonstrated that preferred catheters can achieve the aim of distributing a given volume of injectate over a larger rostrocaudal length of spinal cord, resulting in i) widespread distribution of a small volume of injectate and ii) a dilution of the smaller volume of injectate in larger local volumes of CSF (cerebrospinal fluid), thereby achieving an aim of enhancing distribution and reducing local drug concentration.

With the pattern of slits, internal pressure must be sufficient to first open the valves before injectate is released, enabling small volumes to distribute evenly among the valves. The arrangement behaves as a series of infinitely small holes, whereby, the exit velocity at each valve will be elevated as the transmural pressure increases. Therapeutic agent can advantageously be emitted in single pulse from each valve as the transmural pressure rise to a level which causes the slit vale to open.

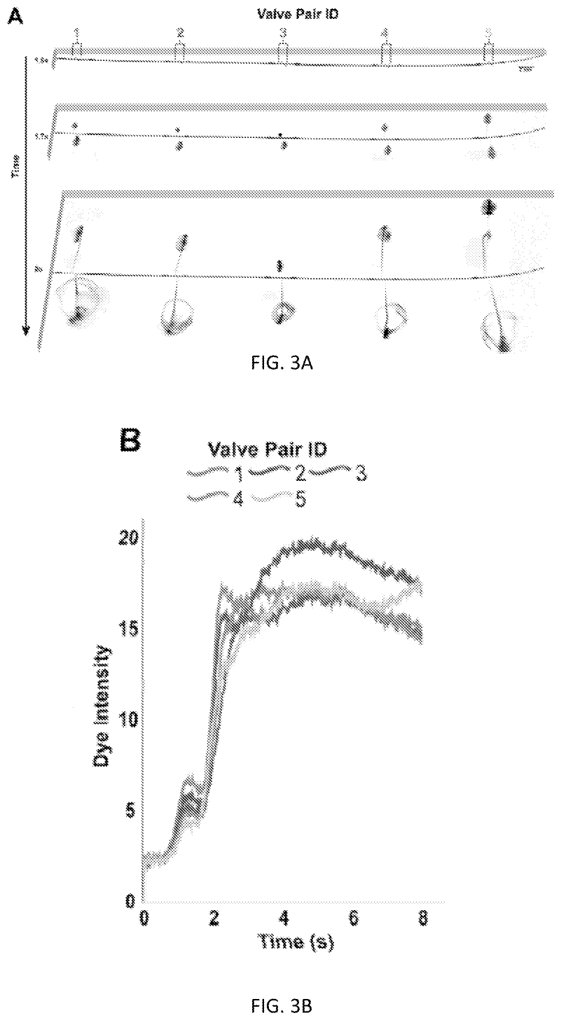

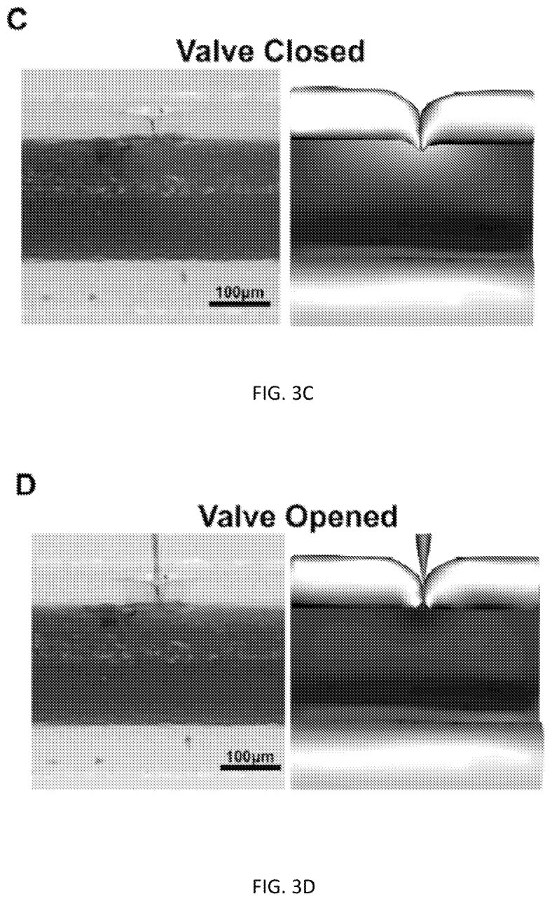

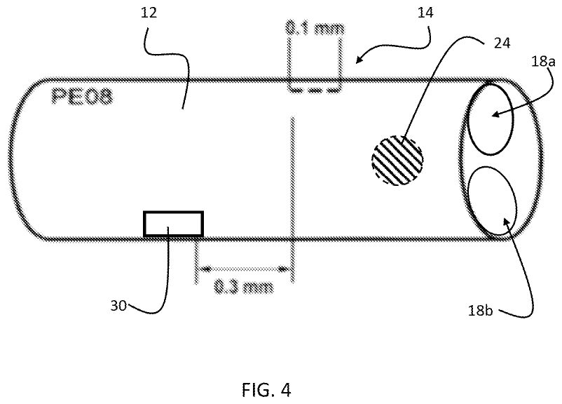

The efficacy of this approach is emphasized in FIGS. 3A-3D, showing blue dye (10 μL) exiting from an experimental paired multi micro valve catheter. The system results in an even distribution of a small volumes of treatment solution over the entire length of the catheter. Further, by pairing such that each valve has a radially offset paired valve, even distribution is provided laterally as well as along the longitudinal length of the catheter.

Another preferred catheter is shown in the partial via of FIG. 4, and includes dual lumens 18a and 18b. The lumen 18a is fluidly connected to a pattern 14 of microvalves as in FIGS. 1A-1C. The lumen 18b interfaces with a pattern of larger ports 30 (one is shown but the ports are distributed longitudinally along the treatment area). These larger ports 30 can be used to remove local CSV by applying suction in the lumen 18b.

### Experiments

Rostrocaudal Dye Distribution: Rats prepared with either conventional open ended (OEC) catheters or the present micro-poly-valves (MPV) catheters received an injection of 7.5 μL of methylene blue dye followed by 12.5 μL saline flush. Animals were sacrificed and the spinal cords were exposed. Injection of new methylene blue dye through a conventional catheter resulted in dark localized staining in the sacral/lumbar cord proximal to the termination site of the catheter. In contrast the present microvalve catheter resulted in a reliable dye distribution in the rostral segments including the cervical cord.

Rostrocaudal distribution of AAV (adeno-associated virus). Intrathecal injection of AAV-9 results in transfection of dorsal root ganglion neurons. Rats prepared with either conventional or present catheters received an injection of 7.5 μL of AAV-RFP followed by a 12.5 μL saline flush. Viral titers of 1e10 or 1e12 viral genomes were injected. One month later bilateral

Dorsal root ganglia (DRG) were harvested at the Cervical, Thoracic, Lumbar, and Sacral levels. At the low titer, both OEC and MPV catheters resulted in robust sacral transfection (e.g. proximal to the catheter tip), but only rats with present MPV catheters had transfection of neurons above lumbar. Even at the highest titer, the conventional catheter injected rats produce relatively modest transfection in contrast to the rats injected with the present microvalve catheters. This reflects the poor rostral distribution of the injectate achieved by the OEC.

Accumulation of debris in the intrathecal catheters over time. Rats with MP V) and OEC catheters were sacrificed after approximately 2 weeks without injection. Decalcification and sectioning of the lumbar spinal column revealed that 5 of 6 OEC catheters displayed significant debris in the catheter. In contrast, the present MPV catheters showed minimal signs of any infiltration and cellular debris reflecting upon the important difference between the closed valved catheter described here in the present invention vs an open ended catheter. This has significant virtue for chronically placed catheter systems which are injected only periodically.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A therapeutic agent delivery catheter system, comprising:
an elongate tube defining a lumen therein formed from biocompatible material and sized to insert into a patient or an animal, the tube having a length that extends from the site of insertion to reach a treatment region of the patient; and
a pattern of slits that penetrates an outer wall of the tube, the slits being small enough to remain closed to contain a solution of therapeutic agent, and being configured to open in response to a pressure pulse in the lumen and emit a microjet of therapeutic agent solution from each slit in the pattern of slits.

2. The catheter system of claim 1, wherein the slits are evenly distributed along a longitudinal axis of the catheter over distance determined by the treatment region.

3. The catheter system of claim 1, wherein the pattern arranges slits at different radial angles around a circumference of the elongate tube.

4. The catheter system of claim 3, wherein the pattern includes adjacent slits that are radially offset from each other.

5. The catheter system of claim 4, wherein adjacent slits are radially offset from each other at an angle of 120° to 180°.

6. The catheter system of claim 1, wherein the elongate tube has a diameter of one of a urethral catheter, a cardiac catheter, a venous catheter, a subcutaneous catheter for delivery to wound margins or perineurally, an intracranial catheter, and a large or microbore intrathecal catheter.

7. The catheter system of claim 1, connected to a port or a pump and control system that is configured to deliver one or more pressure pulses sufficient to open the slits.

8. The catheter system of claim 1 wherein the tube is sized for intrathecal treatment and the pattern arranges slits along a rostrocaudal length.

9. The catheter system of claim 1, comprising a rounded distal tip, wherein the rounded distal tip comprises a distal slit.

10. The catheter system of claim 9, wherein the distal slit is smaller than the slits in the pattern of slits.

11. The catheter system of claim 1, comprising a second lumen and ports along the second lumen, the ports being openings sized to remove local fluid from the treatment region.
